(19) United States
(12) Patent Application Publication
(10) Pub. No.: US 2008/0226496 A1
(43) Pub. Date: Sep. 18, 2008

(54) Systemic Method For Proximity Hygiene and Device With Low-Temperature Sanitizing Chamber in Particular For Food Products

(76) Inventors: Jacques Rivkine, Geneve (CH); Pierre Hennuyer, Veyrier (CH)

Correspondence Address: BACHMAN & LAPOINTE, P.C., 900 CHAPEL STREET, SUITE 1201, NEW HAVEN, CT, 06510, US

(21) Appl. No.: 11/908,827

(22) PCT Filed: Mar. 15, 2006
(86) PCT No.: PCT/FR2006/000582

§ 371 Date: Oct. 5, 2007

(30) Foreign Application Priority Data
Mar. 18, 2005 (CH) 467/05

(51) Int. Cl. A61L 2/10; A61L 2/20; B01J 19/00

(52) U.S. Cl. 422/24; 422/29; 422/242

(57) ABSTRACT

The invention concerns a fast systemic method for low-temperature (athermal) sanitizing in confined chamber, under modified atmosphere, in turbulent conditions by combined production of a highly germicidal ionic chain acting simultaneously on the hydric flux (water) and the aeraulic flux (air), operating under controlled temperature, for killing pathogenic germs and toxins.

The present invention relates to a method and to a device in the food safety and hygiene field.

The subject of the invention relates to a method for proximity hygiene and a device comprising a low-temperature (athermal) sanitizing cell, which integrates a systematic method comprising nanotechnology in a confined chamber, with a restricted volume, under a modified atmosphere and under turbulent conditions, the aim of which is the rapid destruction of pathogenic microorganisms, molds, spores or toxic contaminants, based on the direct, simultaneous and combined reaction of aqueous (water) and gaseous (air) fluid oxidizing agents, which is applied, for food safety, to safety treatments by surface contact, i.e. in particular food products, including fresh (fruit and vegetables), converted, raw or cooked products, food products before or after freezing, or other surfaces and components requiring a safety treatment. This biological method for inactivating mircroorganisms and pollutants extends the freshness of food products by preserving, by sanitization, their organoleptic and nutritive properties. This method reduces reaction times and replaces chemical disinfectants of the fungicide and microbicide class, including chlorinated compounds, and constitutes an alternative to ionization by irradiation.

As regards the prior art of methods related to food hygiene and safety, it has been considered, up until now, that the most conventional techniques for preserving foods are those of pasteurization and sterilization, the aim of which is the destruction or the irreversible inactivation of all microorganisms. These heat-based methods require the use of incubators, autoclaves or ovens with temperatures above 121° and time periods lasting up to one hour. New athermal preservation techniques have emerged, namely: pulsed light, pulsed magnetic fields and pulsed electric fields. The principle of these treatments varies greatly from one method to the other, but the objective of their common denominator is to obtain a rapid reduction of microorganisms at moderate temperatures.

In terms of food preservation, the irradiation process is in particular known. Preservation by irradiation, also known as ionization, involves exposure of the foods to energy originating from sources such as gamma-rays, X-rays or electron beams. During the irradiation, the foods are not heated, as is the case for microwaves. A high-pressure hydrostatic technique has recently been applied for carrying out the microbiological decontamination of foods packaged in flexible packaging, but the installations are rare due to their high cost.

As regards freeze-drying, this is a mild desiccation method which consists in gradually removing the water from a previously frozen product by passage to the vapor phase (sublimation).

In terms of gaseous treatment, techniques for decontaminating air flows by dust removal or filtration, combined with an ultraviolet or chemical product method, are mainly known. It is recognized that foods in contact with air can undergo physical, enzymatic, microbiological and biochemical deteriorations. Modified atmosphere packaging (MAP) is a method which consists in packaging the food products with a gas or a mixture of gases having certain protective and reactive properties (bacteriostatic and fungistatic CO₂). In the proximity hygiene sector, the clean room application, in which the sterility of said clean room is obtained by means of a laminar flow which protects against the introduction of contaminated air and continually treats the air content of the room, is known.

The limiting of fungal and bacterial attacks, in terms of post-harvest treatment of fruit and vegetables, is still based mainly on the SO₂ (sulfur dioxide) fumigation technique and the use of washing water with concentration of active chlorine (bleach) or of chlorine dioxide. Thermal fogging is a variant which combines the effects of a high-speed air flow and of a high temperature, which results in the emission of a fine fog of microparticles of biocidal products. It should be noted that, pursuant to the European Regulations 2002/2003, the use of sodium hypochloride and of chlorine in contact with foods will be prohibited in 2006 in all countries of the European Union, in particular for category IV: prepacked fresh ready-to-use products.

The importance of refrigeration and of freezing is recognized among post-harvest preservation treatment technologies. The most advanced pulsed-air cooling systems use powerful fans for blowing refrigerated air. Most of the methods require long reaction times before the inactivating effect on the bacterial pollutants is obtained.

Studies and research are ongoing regarding oxidation with ozone O₃ (aq). “Advanced oxidation” processes (AOPs) allow complete degradation (mineralization), in an aqueous medium, of organic molecules which are toxic to humans and to the environment. The effects of ozonation are in particular known in water treatment with treatments of 5 to 10 mg per m³ (Evaluation of ozone for airborne and surface disinfection—Campden & Chorleywood Food Research Association Group). The pollution created by organic compounds has led to the development of research on advanced oxidation processes (AOPs). AOPs comprise processes where an oxidant and irradiation are combined (H₂O₂/UV, UV/O₃, O₃/UV/H₂O₂), but also in vacuum ultraviolet (V-UV) irradiation processes, with wavelengths of less than 200 nm. Also included among AOPs is photocatalysis, which combines visible UV radiation and a photocatalyst, generally a semiconductor. The most commonly used is TiO₂ because of its oxidizing and reducing properties and the fact that it is completely innocuous. Studies on advanced oxidation processes generating hydroxyl radicals are being carried out by various universities and research centers, including, in Switzerland, EAWAG. They are mainly directed toward the treatment of water:
H₂O₂/UV, O₃/H₂O₂ and O₃/UV processes,
Fenton and photo-Fenton processes,
TiO₂/UV process with or without H₂O₂.

The most recent developments in food safety concern the use of a lactoperoxydase system as technological auxiliary for the treatment of salads (category IV: prepacked fresh ready-to-use products). The principle consists of the formation of isothiocyanate ions (OSCN⁻) having an antimicrobial action, which are produced by passing water enriched in potassium thiocyanate and hydrogen peroxide over a reactor containing lactoperoxydase attached to a support (particles of clay) . In the field of applications in the hospital environment, a solution for sterilization with ozone has been designed for the sterilization of surgical instruments (TSO₃) and approved by the United States Food and Drug Administration.

Moreover, the use of turbulent conditions in processes applicable to humidification in order to obtain a vapor consisting of very fine droplets is recognized. Mention may be made of various patent applications for sanitization intended for agricultural products, using high concentrations of ozone, oxidizing fluids or aqueous solutions sprayed in a chamber. The following patents are known: WO 2004010798 (sanitization), WO03034831 (elimination of pathogenic agents), WO 03017773 (preservation of produce), WO02071852 (storage device), EP1254600 (preservation method), WO0238467 (fungicide in a controlled atmosphere chamber), EP136940 (system for using microbicidal agents), EP1388290 (controlled temperature chamber).

Other patents of the prior art can also be mentioned, for instance:
Patent EP0341069 applies to a general method of sterilization, in particular for packaging. It uses the UV source as a direct source for sanitizing microorganisms using ozone decomposition by irradiation so as to form an oxygen radical. The invention distinguishes itself by virtue of a system characterized by separation of the aqueous and gaseous treatment, including a capacity for a high concentration of the O₃ oxidant in the aqueous fluid, before germicidal release in a pressurized indirect phase, in a Lagrangian turbulent phase, and pressurization of the treatment chamber allowing optimization of the sanitizing effect on the surfaces, and a considerable reduction in the potencies of the oxidizing sources to be used and in the cost of implementation of the method.
Patent application US 2004/0231696 discloses a device comprising a two-compartment chamber for the elimination of pathogenic and toxic agents from surfaces via the direct principle of photochemical reaction or ozone, a system concerning exclusively the treatment of air and filtration thereof. As mentioned for patent EP 0341069, the invention distinguishes itself by virtue of an aqueous and gaseous system in a one-cell assembly characterized by a pressure Pa above the outside atmospheric pressure and turbulence.
International patent application WO 03/080127 discloses a method for spraying an antibacterial liquid chemical agent in a chamber, through nozzles, applicable to fresh food products. The invention distinguishes itself by virtue of its own system for the production of oxidation, which does not leave any chemical trace or any toxic residue on the treated foods, since the residue of the germicidal fluid is converted to oxygen.
Patent GB 444007 applies to the storage of food products, paper and tobacco, for protecting them during their storage against deterioration due to bacteria by spraying an aqueous solution with a mineral salt (NaCl) content in a refrigerating chamber, which is negative, including UV-ionization of the air. This invention distinguishes itself by virtue of its sanitizing inoculation capacity without modification of the organoleptic properties and its capacity for replacing chemical disinfectants, including water-based compounds with a sodium content which are prohibited pursuant to European Regulations 2002/2003, in particular for converted fresh food products (category IV: prepacked fresh ready-to-use products). According to a particular advantage, the invention allows storage at a positive temperature, in the knowledge that a negative temperature induces side effects that can be damaging to products such as fruit and vegetables.
International patent WO 99/20129 concerns a complete system of disinfection for animal products by exposure to a gas derived from an aqueous solution with a high salt (sodium) content, subjected to an electrolysis device. This system requires, for its process, a washing container, an additional treatment area and a third area for packaging. This patent constitutes a variant of the patent WO 444047. The invention distinguishes itself by means of a method in a one-cell chamber, and by the use of pure service water without any particular characteristic or pH indication; the oxidation treatment does not induce any conversion in terms of taste, nature or color of the treated foods nor residues.
Patent application US 2004/0156959 relates to a modular, adjustable tunnel that can be assembled and that comprises UV-radiation equipment for producing ozone, hydroperoxides and hydroxyl radicals for the purposes of sanitizing foods. The invention distinguishes itself by virtue of the confined shape of the treatment chamber, which has a predetermined volume, and by virtue of its dual system of disinfection with an oxidizing aqueous and gaseous fluid, completed by a device for a controlled atmosphere, under turbulent conditions. These parameters are not reproduced by application US 2004/0156959 and the inventive principle of juxtaposition of the chambers for direct exposure of the foods is not a continuous tunnel in nature.
Patent FR 1524290 concerns a treatment by ripening and drying of products of animal or plant origin in a chamber. The aim of the process is to increase the moisture by means of a liquid product combined with intensive circulation of a gas at a rate of between 1 and 5 milliseconds, the gas circulating in the form of a primary circuit and a secondary circuit by means of directional fans. As stated in its description, the treatment does not comprise any dual aqueous or gaseous oxidation treatment having an oxidizing capacity. The continuous-flow ventilation system is very different from the means used and described in the invention. The invention is based on the pressurization and the regulation of the generation of turbulence, in chaotic and Lagrangian form, of the volume of the treatment chamber, with a capacity for regulation and optimization of the force applied to the flow of the fluids, to their introduction (positive) or to their flow (negative).
Patent application EP 1483972A1 concerns a method for preserving the quality of agricultural products, such as fruit, vegetables, flowers and plants, in a gas atmosphere in a closed chamber under turbulent flow conditions. The method is based on exposing the air under turbulent conditions to a photocatalytic oxidation process using rays from UV lamps. Ozone is released as a simple gaseous chemical substance, without a capacity for direct oxidation on the surfaces, and the exposure is conditioned by a forced passage of air flow through obstruction means. The cold source between 0 and 15° C. is adjusted according to the ripening of the fruit or vegetable. The invention is based on regulation of the refrigeration at a positive temperature between 7° and 15° C., in a pressurized and turbulent chamber as common elements for effectiveness of the oxidation process, in the knowledge that the water-solubility of the molecules (O₃ electron) and the effectiveness of their depolarization during vaporization depend on the ambient temperature.
International patent application WO 02/05665 uses a gaseous oxygen-based fluid comprising oxidizing radicals, of the ozone type, in a proportion of 50% and maximum 5% by weight, for reducing and eliminating toxic chemical substances or other components such as microorganisms, on organisms before freezing. This method is based on the principles of the reactivity of oxygen by depolarization of the O₂ molecule and conversion thereof to the O⁰ or OH⁰ radical. It uses a plant resistant to pressures of 200 mbar to 400 mbar. It is recognized that oxygen carries risks of ignition and explosion which require strict safety measures and which are contrary to the principle of the invention. The exposure as defined in a variant in an aqueous concentration for 12 to 14 minutes has no effect given the instability of the molecule. The invention has identical surface treatment application characteristics, it distinguishes itself by virtue of application of the aqueous and gaseous advanced oxidation processes by catalyzation of the O₃ molecule to negative OH ions, and pressurization of the treatment cell within the limits of 400 Pa under turbulent conditions, so as to obtain complete diffusion of the oxidizing agent within an effectiveness period of 2 to 4 minutes. The invention is improved by means of a system of vectorization with a rotating spiral shaft which allows the essential exposure, to the aqueous fluid, of the complete surface and of the substances such as grains, seeds or products, including those denoted in patent application WO 02/05665.

The invention is distinguished from prior art which has just been cited in that it comprises a treatment cell for obtaining a systemic nanotechnology sanitizing method, using a high concentration of negative OH free radical oxidants, in a confined chamber, under a modified atmosphere and under turbulent conditions. According to this mode, the invention results in a process for disinfecting the surfaces of food products and for controlling the quality of the air and of the water in a restricted volume, in very rapid reaction times, even under bactericidal and viricidal conditions (for OH: 0.3 to 05 mg/l for four minutes in comparison with chlorine: 0.1 to 0.2 mg/l for 30 to 45 minutes or chlorine dioxide 0.1 to 0.2 mg/l for 30 minutes).

The invention applies, for example, to (food-producing) plant production, for eliminating microorganisms and contaminating agents which induce spoiling and molds which attack the collected products, i.e. for the most well known: alternarium black rot, black rot, soft rot on perishable goods, white mold, brown stain, gray mold, blue mold, black mold, fusarium bulb rot, fusarium rot, gangrene and listeriosis.

An object of the invention is also to be applicable to the sector of the collective restoration or distribution after preparation of raw or grated, cooked and ready-for-use products, pasta and rice, fillets of fish, or meats, as a solution to the prevention of the risks of collective toxico-infections (collective food toxico-infection) through microbial contamination and sporing—aerobic microorganisms—salmonellosis—listeriosis—botulism—chemical contamination—E. coli Ddt—pesticides—mercury—dioxin—natural toxins—molds and yeasts.

According to one embodiment which is specific to the invention, the latter constitutes a nontoxic bioecological solution which does not produce any undesirable derivatives since the residue from the germicidal flow is converted to oxygen. The application of the method has no effect on human health and does not modify the quality of the products or the demand for them or their organoleptic properties. Compared with the known methods, it reduces the reaction times and is a replacement for chemical disinfectants of the fungicide and microbicide class, including chlorinated compounds. It is an alternative to the method of ionization by irradiation.

In order to achieve these results, the operating principle of the invention is based on the implementation of a fast systemic method for low-temperature (athermal) sanitizing in a confined chamber, under a modified atmosphere and under turbulent conditions, by the combined production of a highly germicidal ionic chain acting simultaneously on the aqueous (water) flow and the gaseous (air) flow, operating under controlled temperature, for killing pathogenic microorganisms and toxins.

The embodiment is based on the catalyzed production of gas in cooled water at a positive temperature and ultraviolet radiation acting on the air, the whole having an electrophysical oxidation effect through generation of OH⁻ negative ions, and a series of ion exchanges, polarization and depolarization, the whole under turbulent conditions.

According to an inventive characteristic of sanitizing method through the method of pressurizing the treatment chamber, the amplitudes are not constant but generate a wave turbulence. The result obtained is an absolute dispersion of the germicidal gaseous and aqueous flows, making it possible to save on implementation means. The pressurizing of the treatment chamber has the effect of maintaining and assisting the generation of turbulent conditions. The result is a reduction in operating times and the exclusion of the risks of intrusion of dust and repollution. In this way, complete diffusion of the oxidizing products acting within surrounding limits under integrated control is obtained, while at the same time managing the parameters (No. x to Z). This principle is applicable to products of any type that must be introduced into the treatment chamber provided that the data (No. x to Z) are repeated.

Currently, it should be taken into consideration that research on the use of ultraviolet and ozone has been mainly carried out in the United States and in Canada. The Journal of Food Science reports that exposure to UV-C is found to be a very advantageous approach for facilitating access to the market and the distribution of fresh fruit and vegetables.

This treatment slows down the ripening process (strawberries which remain firm for a longer period of time). At the useful dose, it is considered that ultraviolet rays have beneficial effects. Finally, the use of nonradioactive light does not pose a problem for consumers.

The invention provides a solution through the capacity for sanitized packaging of fresh or converted food products of category IV (prepacked ready-to-use products), capable of considerably increasing the storage time of various food products without resorting to refrigerating plants, for example freezing plants. According to the embodiment of the invention, it is considered that subjecting fresh fruit and vegetables to the proximity hygiene method, as described, extends their shelf life by a third. The Centre de Recherches d'Horticulture Canadien [Canadian Horticultural Research Centre) has observed that freshly picked strawberries stayed fresh for approximately 14 to 15 days. It is acknowledged that treatment with a freezing plant results in negative side effects, such as, for example, browning of fruit surfaces. According to a specific advantage of the invention, the method reproduces the effects of natural physiological conversion in a systemic and concentrated form in the implementation, without any risk of impairment or of side effects.

The basis of the invention is the result of the derived application of nanotechnology and Lagrangian turbulence physics. It is based on the use and the organization of components which follow from the known prior art. The systemic application and the organization of these simultaneous, synchronized and controlled components, in the form of a configured assembly, constitute an important modification compared with the known techniques. It is an innovative approach in terms of low-temperature (athermal) proximity hygiene technique related to the decontamination and preservation of agricultural harvest, fresh, raw or cooked foods and food products to be frozen or thawed and also to the packaging processes which are related thereto.

The invention results in hygiene-related and economic advantages such as:
long-lasting elimination of bacterial and toxic agents, including destruction of spores on food surfaces (aerobic microorganisms, salmonellae, legionellosis, listeriosis, etc.);
extension of the shelf life of fruits and vegetables and of the sanitized packaging thereof, including, by extension, food products in general (dairy products, fish);
reduction of the chemical hazards and biological risks that can induce poisoning in fresh, frozen or cooked products;
reduction of post-harvest losses for sensitive vegetables and fruits, which can reach 40% to 60% of harvests;
the sanitized products cooled to a positive temperature of 8° are able to be placed in protective packaging and to reach the consumer without any further risk of pollution;
the method does not modify any of the product quality parameters; it extends the life of the products and their freshness, and maintains their taste and all their organo-leptic qualities without any impairment;
the method falls within the recommended HACCP (Hazard Analysis and Critical Control Point) measures advocated for foods and at times of packaging or conversion;
the advantages of the invention can be felt in terms of ecology and long-lasting management benefits;
market gardeners, fruit growers and commercial farmers are the direct beneficiaries of the improvement in quality that comes from the possibility of better ripening.

Other capabilities are applicable to the dairy and cheese agrofoods industries.

The decrease in post-harvest losses is added to by the impact of a reduction in the “wastage” of rejected food products whose storage date has expired at the distributors. Ecologically, the use of bactericidal or fungicidal chemical preservatives is avoided.

The method of the invention results in a saving in terms of plant and running costs if the proximity hygiene method disclosed is compared with known modified atmosphere, pulsed light, irradiation, ultrahigh pressure and refrigerated chamber plants. The invention constitutes an alternative in terms of rationalization and economy of investment costs. Since the method is mobile, it can be set up in proximity to the production sites.

It is acknowledged that commercial farmers suffer considerable post-harvest losses due to the rapid development of contaminants and toxic products which impair the products and make them unfit for the market. It is acknowledged that the washing water used by commercial farmers does not always correspond to health standards and that it is an important agent of pathogenic contaminations and poisonings. It is acknowledged that the bacterostatic application of cold conditions slows down cell division, but does not eliminate bacteria, and that frozen bacteria again placed at ambient temperature develop more rapidly.

The invention provides a new dimension to the quality and the innocuousness of foods and constitutes progress by virtue of an economically accessible process cost. The economic advantage obtained by virtue of the invention relates to the application of a method of decontamination by nebulization of water and, in parallel, by the creation of turbulence in and pressurization of the treatment chamber, contributing to a considerable reduction in volume of the water to be treated (1 1/H per m³), which is itself reflected by an advantageous proportioning of the power of the hydrological oxidant generator and, consequently, favorable manufacturing and running cost factors.

The advantages of the invention are also expressed by the explicit characteristic of the separate use of aqueous and gaseous oxidant generators. According to the gaseous treatment embodiment, the cell lends itself, as sanitized chamber placed in an atmosphere that is specific to packaging operations, to hygiene procedures for veterinary operative treatments. In terms of the safety of individuals, it is specified that no human intervention is necessary inside the chamber during the treatment phases and that this constitutes an innovative provision in terms of proximity hygiene.

The present invention also relates to the features of a device which will emerge over the course of the description which follows, and which should be considered in isolation or according to any of their possible technical combinations.

This description, given by way of nonlimiting example, will make it possible to understand more clearly how the invention can be implemented with reference to the attached drawings in which:

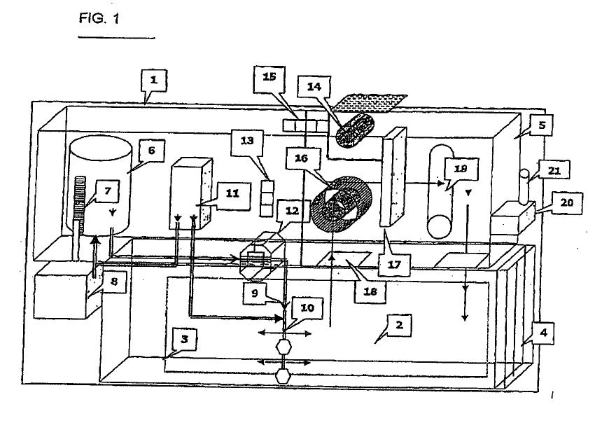

FIG. 1 specifies the sanitizing cell plant and the detailed organization of its components.

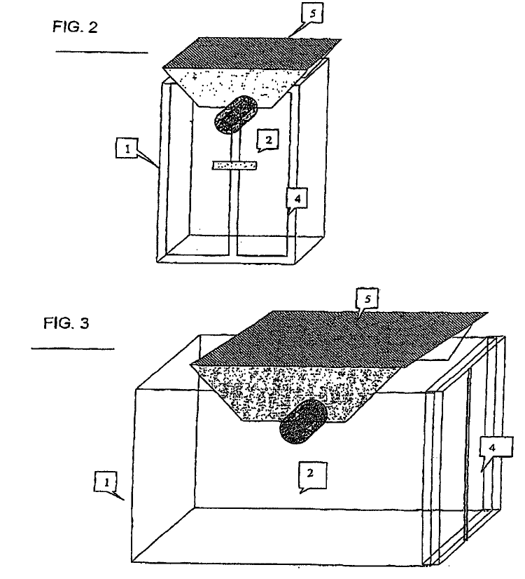

FIG. 2 relates to an overall view of an embodiment in the form of a cabinet, for application in market gardening product restoration or storage.

FIG. 3 relates to an overall view of an embodiment in a one-cell form, for application in rural production and treatment of harvests.

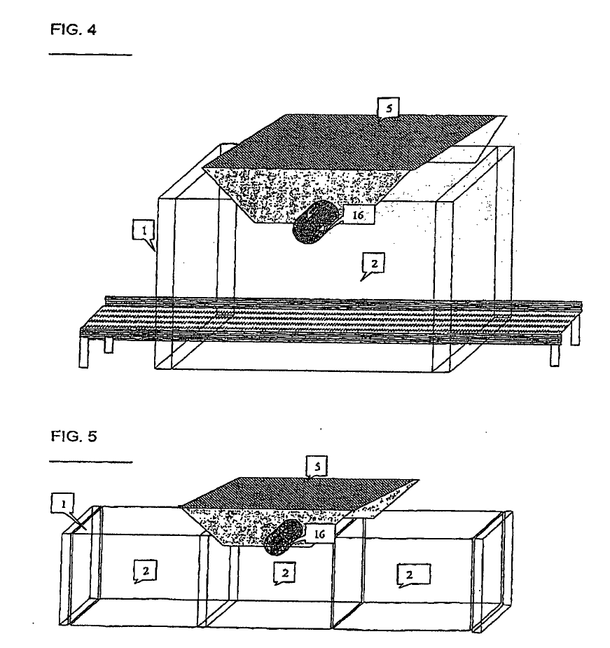

FIG. 4 represents an overall view of an embodiment in the form of a cell with a conveyor-belt feed system.

FIG. 5 represents an overall view of an embodiment with the juxtaposition of several cells, for applications in parallel and bulk treatment.

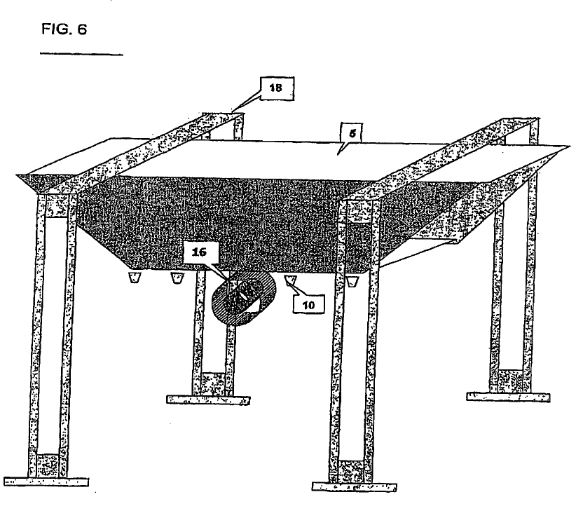

FIG. 6 represents the organization of the components in the form of a mobile kit that can be integrated into an existing site.

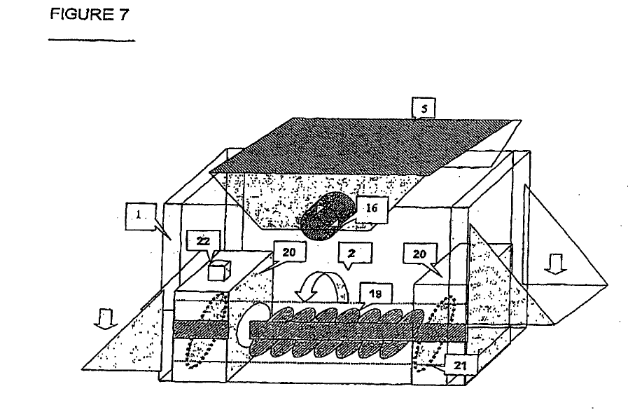

FIG. 7 specifies the specific plant of the sanitizing cell with the detailed mechanisms for exposure to the treatment and continuous vectorization of the products to be treated.

The functioning specific to the invention is described hereinafter by means of the picture and references of FIG. 1. The crude or cropped fresh products (vegetables, fruit, or the like) intended to be decontaminated are placed in the sanitizing cell 1, and more specifically in the treatment chamber 2. The treatment chamber is designed in a confined shape, coated with a leaktight protection or membrane 3 equipped with leaktight doors 4 and placed under a controlled atmosphere. The equipment for production of the gaseous and aqueous flows are grouped together in a compact technical module 5 which is fixed or mobile depending on the embodiment selected, bringing together a treatment chamber 2 of the confined type and a leaktight membrane 3.

The inside air volume of the treatment chamber 2 is continuously filtered with a particle filter 17 and sanitized by irradiation produced by a two-wavelength gaseous germicidal radiation generator 19.

In the initializing phase of the method, the atmosphere of the treatment chamber 2 is placed under a positive pressure relative to the pressure of the air outside the box. This increase in pressure is obtained by the intake, by means of an axial turbine 14, of a necessary and sufficient volume of outside air. It is a question, by applying of the physical principle of confined and parameterized flows, in the multifractal structure of the dissipation field at the boundary between the inertial system and the dissipating system, of the relative atmospheric pressure being set at Atm.+1 relative to the pressure of the air outside the treatment chamber, this being throughout the treatment phase in Lagrangian formalism. This element allows optimal integrated dynamics, creating a saving in terms of means used.

The pressurization of the treatment chamber 2 and the generation of gaseous turbulence therein are obtained by means of an axial turbulence turbine 16 which recycles the gaseous fluid via a recycling point for intaking air 18 and eliminates the airborne particles (spores, etc.) through a particle filter 17. The parameters for placing the inside of the treatment chamber 2 under atmospheric pressure are controlled by a gas regulator 15 comprising a flowmeter, a pressure-sensitive switch and a solenoid valve.

The content (fruits, vegetables, packaging) of the treatment chamber 2 is brought into contact with a highly germicidal oxidizing agent produced by a generator of oxidizing agents (negative OH free radicals) 11. In this embodiment, the free radicals are, by means of connecting tubing 9, directly injected into a water treatment reservoir 6 in order to eliminate the pathogenic agents and to saturate the aqueous solution with OH⁻ radicals.

The water is pre-cooled to a positive temperature of 5 to 10° C. by a cooling exchanger 7 connected to a refrigerating unit 8, in order to obtain a low-temperature aqueous product. The oxidizing fluid is nebulized by a pressurized circulating pump 12 in the chamber by means of a vaporization system obtained through nebulizing heads (micronozzles) 10 placed around the treatment chamber 2. The pressure nebulization obtained from the water treatment reservoir 6 brings about a drop in pressure of the order of 1 At. (for a maximum outside temperature of 25° C.) which is compensated for by the axial pressure turbine 14 controlled by a device 21 that senses the outside atmospheric pressure. The microbiological inactivation time parameters correspond to a time or a volume determined by the water regulator 13 comprising a flowmeter and a pressure-sensitive switch.

The technical diffusion devices 6-7-8-9-10 can be used for the diffusion, in the treatment chamber 2, of any type of oxidizing aqueous or gaseous product of the ionic, gaseous or chemical type.

The device according to the invention comprises a control panel 20, a gas regulator 15, a water flow regulator 13 and an external atmospheric sensor 21, for controlling the microbiological inactivation treatment cycle (times and flow rates) and the maintenance of the cell jointly under a modified atmosphere and turbulent conditions.

This plant allows the combined production of a highly germicidal ionic chain and makes it possible to expose the contents jointly to two effective (gaseous and aqueous) sources for sanitizing surface pathogen microorganisms, toxins or molds and to eliminate the risks of recontamination by sporulation. According to the invention, the procedures inside the treatment chamber (cell) are carried out without human intervention and the treatment cycles are managed and controlled from a control/regulation panel 20 attached to the compact technical module 4. By means of this original configuration for controlling the germicidal oxidation generators 11 and the axial turbulence turbine, individual and independent actuation is possible, for example for making polluted water drinkable and more advantageously for maintaining the content of the chamber under a modified atmosphere for various drying, storage, preservation or packaging procedures.

According to one embodiment, the method advantageously applies to the treatment of products in the form of grains or chopped products through the continuous system of mechanized introduction (FIG. 7), by means of one or more spiral conveyors 19 which produce an effect of turning and exposing the surfaces X to be treated through 360°. The system is particular in that it has at its inlet a loading chute, at the center a confined treatment space under turbulent conditions 16 according to the stated principles, and conveyance to the outlet of the operating cycle via a discharge chute.

Sealing of the oxidation fluid is provided at the inlet and at the end by housings 20 equipped with a set of disks with spiral seals 21 and an ozone residue destroyer 22 identical to the standard model of the treatment chamber.

By virtue of the device of the invention, it is possible to realize embodiments for the following specific applications:
in the form of cabinets (FIG. 2) for use in restoration,
in the form of a single cell (FIG. 3) for application in a rural production area,
in the form of a tunnel fed via a conveyor (FIG. 4),
in multicellular form (FIG. 5) for agroindustrial applications for the treatment of large volumes,
as an independent mobile kit (FIG. 6) comprising the compact technical module 4, with integration of the nebulizing heads 10 and support feet 17, for operation in a closed chamber or under a tarpaulin, in a method of application for the decontamination of instruments or for a hygiene procedure (veterinary procedures),
in the form of a box with continuous vectorization (FIG. 7) by means of a spiral shaft feed mechanism 19 for a continuous application, for seeds, grains or converted products referred to as category IV (prepacked fresh ready-to-use products).

In summary, the subject of the invention relates to a method for proximity hygiene and a device comprising a low-temperature (athermal) sanitizing cell, which integrates a systemic method comprising nanotechnology in a confined chamber, with a restricted volume, under a modified atmosphere and under turbulent conditions, the aim of which is the rapid destruction of pathogenic microorganisms, molds, spores or toxic contaminants, based on the direct, simultaneous and combined reaction of aqueous (water) and gaseous (air) fluid oxidizing agents, which is applied, for food safety, to safety treatments by surface contact, i.e. in particular food products, including fresh (fruit and vegetables), converted, raw or cooked products, food products before or after freezing, or other surfaces and components, such as packagings, requiring a safety treatment. This biological method for inactivating mircroorganisms and pollutants extends the freshness of food products by preserving, by sanitization, their organoleptic and nutritive properties. This method reduces reaction times and replaces chemical disinfectants of the fungicide and microbicide class, including chlorinated compounds, and constitutes an alternative to ionization by irradiation.

The crude or chopped fresh products (vegetables, fruits, or the like) intended to be decontaminated are placed in the sanitizing cell 1, and more specifically in a treatment chamber 2. The treatment chamber is designed in a confined shape, coated with a leaktight protection or membrane 3 equipped with leaktight doors 4 and placed under turbulent conditions and under a controlled atmosphere. The equipment for producing the germicidal gaseous and aqueous flows are grouped together in a compact technical module 5 which is fixed or mobile according to the embodiment selected.

1-16. (canceled)

10. A systemic method for low-temperature sterilization, comprising the steps of: providing technical components arranged in a confined and pressurized chamber, under a modified atmosphere and under turbulent conditions regulated by gaseous waves; placing objects within said chamber; and creating a combined production of a highly germicidal ionic chain by means bf which pathogenic mircroorganisms and toxins are destroyed by acting simultaneously or separately on aqueous and gaseous generators and on surfaces of the objects placed in the chamber.

11. The systemic method for sterilization as claimed in claim 10, further comprising controlling pressurization and regulation of a generation of turbulence, in chaotic and Lagrangian form, of the volume of the chamber, with a capacity for regulation and optimization of a force applied to a flow of fluids, and to an introduction of said fluids or to the flow of said fluids.

12. The systemic method for sterilization as claimed in claim 10, further comprising using a force for concentration of negative OH free-radical oxidizing agents, in the confined chamber, under the modified atmosphere, and under the turbulent conditions.

13. The systemic method for sterilization as claimed in claim 10, further comprising creating a catalyzed gas production in cooled water at a positive temperature and ultraviolet radiation acting on the air, so as to generate an electrophysical oxidation effect through generation of OH⁻ negative ions, and a series of ion exchanges, polarization and depolarization under said turbulent conditions.

14. The systemic method for sterilization as claimed in claim 10, further comprising producing a gas by an oxidizing agent generator, catalyzed in a reservoir of water, and providing a radiation with an electrophyical oxidation effect by generation of negative OH ions, causing a succession of ion exchanges and of polarization and depolarization, without any chemical trace and any toxic residue.

15. The systemic method for sterilization as claimed in claim 10, further comprising placing the atmosphere of the chamber under a positive pressure relative to a pressure of the air outside the chamber.

16. The systemic method for sterilization as claimed in claim 15, further comprising obtaining the positive pressure relative to the pressure of air outside the chamber is obtained by intaking, by means of an axial turbine, a volume of outside air according to a relative atmospheric position.

17. A device comprising a treatment chamber of the confined type and a leaktight membrane.

18. The device as claimed in claim 17, further comprising a compact technical module bringing together, in a single combination, technical components for generating and driving germicidal oxidizing fluids and for generating turbulent conditions, including a water reservoir and cooling.

19. The device as claimed in claim 17, wherein an interior air volume of the treatment chamber is continuously filtered with a particle filter and sanitized by irradiation produced by a two-wavelength gaseous germicidal radiation generator.

20. The device as claimed in claim 17, wherein pressurization of the treatment chamber and the generation of gaseous turbulence therein are obtained by means of an axial turbulence turbine which recycles the gaseous fluid by means of a recycling point for intaking air and eliminates the airborne particles through a particle filter.

21. The device as claimed in claim 17, wherein pressurization of and the generation of gaseous turbulence in the treatment chamber are obtained by means of an axial pressure turbine and an axial turbulence turbine.

22. The device as claimed in claim 17, wherein diffusion of aqueous oxidants is obtained by means of a pressure pump, through nebulizing heads placed in the treatment chamber, under a pressurized atmosphere and under turbulent conditions.

23. The device as claimed in claim 22, further comprising technical diffusion devices for the diffusion in the treatment chamber of any type of aqueous or gaseous oxidizing product of the ionic, gaseous or chemical type.

24. The device as claimed in claim 17, further comprising a control panel, a gas regulator, a water flow regulator and an external atmospheric sensor, for controlling the microbiological inactivation treatment cycle and the maintenance of the cell jointly under a modified atmosphere and turbulent conditions.

25. The device as claimed in claim 17, wherein the device is in a form of cabinets for use in restoration.

26. The device as claimed in claim 17, wherein the device is in the form of a single cell for application in a rural production area.

27. The device as claimed in claim 17, wherein the device is in the form of a tunnel fed via conveyor belt.

28. The device as claimed in claim 17, wherein the device is in multicellular form for agroindustrial applications for the treatment of large volumes.

29. The device as claimed in claim 17, wherein the device is an independent mobile kit comprising the compact technical module, with integration of the nebulizing heads and support feet, for operation in a closed chamber or under a tarpaulin, in a method of application for the decontamination of instruments or for a hygiene procedure.

30. The device as claimed in claim 17, wherein the device is in the form of a box with continuous vectorization by means of a spiral shaft feed mechanism for a continuous application concerning seeds, grains or category IV converted products.
